(19) United States
(12) Patent Application Publication
(10) Pub. No.: US 2013/0295587 A1
(43) Pub. Date: Nov. 7, 2013

(54) METHOD OF COUPLING BINDING AGENTS TO A SUBSTRATE SURFACE

(71) Applicant: Hans Sjobom, Uppsala (SE)

(72) Inventor: Hans Sjobom, Uppsala (SE)

(73) Assignee: GE HEALTHCARE BIO-SCIENCES AB, UPPSALA (SE)

(21) Appl. No.: 13/980,649

(22) PCT Filed: Jan. 20, 2012
(86) PCT No.: PCT/SE12/50047

§ 371 Date: Jul. 19, 2013

(30) Foreign Application Priority Data
Jan. 24, 2011 (SE) 1150040-2

(51) Int. Cl. G01N 33/543; B05D 3/00

(52) U.S. Cl. CPC G01N 33/54366; B05D 3/002
USPC 435/7.1; 422/69; 436/501; 435/287.2; 427/301

(57) ABSTRACT

A chemical modification method of a defined area on a substrate surface, comprising the steps of:
a) providing a flow cell having a reactive substrate surface,
b) providing a laminar flow of a first fluid comprising a first reagent and a laminar flow of a second fluid comprising a second reagent, adjacent to the flow of the first fluid, such that the two laminar fluids flow together over the reactive substrate surface with an interface to each other and,
c) dynamically controlling the relative flow rates of the first and second fluids to position the interface so that a predetermined area of the reactive substrate surface is repeatedly exposed to both the first and the second fluid to obtain a chemically modified predetermined area of the substrate surface.

### TECHNICAL FIELD OF THE INVENTION

The present invention relates to a method of coupling binding agents to a substrate surface by passing a binding agent-containing fluid flow over the surface, and more particularly by using hydrodynamic addressing techniques to selectively direct fluid flows to desired surface areas. The invention also relates to analytical use of the method.

### BACKGROUND OF THE INVENTION

Flow cells are used extensively nowadays in a variety of analytical systems.

Typically, the flow cell has an inlet opening, a flow channel with a sensing surface, and an outlet opening. A sample fluid to be investigated is introduced through the inlet opening, passes through the flow channel and leaves the flow cell through the outlet opening. The flow cell may have more than one inlet opening and optionally more than one outlet opening to permit desired manipulations of the flow pattern within the flow cell. The sensing surface usually comprises a substance layer to which a recognition element for an analyte in the sample is immobilised, typically a biochemical affinity partner to the analyte. When the analyte interacts with the recognition element, a physical or chemical change is produced on the sensing surface that can be detected by a detector, e.g. an optical, electrochemical or calorimetric detector. A flow channel may contain two or more sensing surfaces with different recognition elements.

The sensing surface or surfaces in the flow cell may be functionalized, or activated, in situ, i.e. within the flow cell. WO 90/05305 discloses a method for functionalising a sensing surface having functional groups thereon by passing a reagent solution containing a bi-or polyfunctional ligand over the surface, the ligand having a function which immobilises the ligand on the sensing surface and at least one more function which is exposed on the sensing surface for interaction with the analyte. In a specific embodiment, the sensing surface has a bound carboxymethyl-dextran layer.

After activation of the surface through derivatisation with N-hydroxysuccinimide (NHS), mediated by N-ethyl-N′-(dimethylaminopropyl) carbodiimide (EDC), a ligand in the form of aminotheophylline or aminobiotin is coupled to the activated surface.

WO 99/36766 discloses methods and systems using hydrodynamic addressing techniques to allow immobilization of different ligands to predetermined sensing areas within a single flow cell channel, as well as to permit controlled sample delivery to such functionalized areas. For a Y-type flow cell, which has an inlet end with two inlet ports and an outlet end with one outlet port, and a sensing surface between the ends, WO 99/36766 describes functionalization of two spaced apart sensing areas with different ligands. This is done by providing a laminar flow of a coupling fluid adjacent to a laminar flow of non-coupling (blocking) fluid such that the fluids flow together over the sensing surface with an interface to each other. By adjustment of the relative flow lo rates of the two fluids the interface may be positioned laterally such that activating fluid selectively contacts a desired area of the sensing surface. More specifically, if the coupling fluid initially contains a first ligand capable of binding to the sensing surface and the interface is positioned such that the coupling fluid covers, say, about one third of the lateral extension of the sensing surface, and the blocking fluid covers the remaining two thirds, the first ligand will be immobilized to the first-mentioned third of the sensing surface. Then, the coupling fluid is replaced by blocking fluid, and the blocking fluid is replaced by a coupling fluid containing a second ligand. By positioning the interface such that the coupling fluid again covers about one third of the lateral extension of the sensing surface, now, however, at the opposite side of the flow path, the second ligand will be immobilized to that area, thereby providing a sensing surface which, as seen laterally, has about one third immobilized with the first ligand, about one third immobilized with the second ligand, and an intermediate non immobilized third which only has been in contact with blocking fluid and may suitably be used as a reference area.

WO 03/102580 describes functionalization of more than two spaced apart sensing areas with different ligands in a Y-cell, using hydrodynamic addressing techniques with coupling protocols where each successive coupling of a binding agent to a substrate area is followed or preceded by selective deactivation or activation of a selected area or areas. When more than three sensing areas are functionalized with these methods, at least one ligand will have to flow over a sensing area where a previous ligand has been immobilized. This leads to a certain degree of immobilization of the new ligand together with the previous one and a consequent loss in selectivity of the analysis. Accordingly there is a need for improved methods of coupling using hydrodynamic addressing techniques.

### SUMMARY OF THE INVENTION

One aspect of the invention is to provide a highly selective chemical modification of defined sensing areas on a substrate surface. This is achieved with a modification method as defined in claim 1.

One advantage with the method is that multiple defined areas can be selectively functionalized with different ligands, without cross-talk between the different areas. Further advantages are that the areas can be activated with mixtures of mutually reactive activation reagents without degrading side reactions and that they can be functionalized with pH-sensitive ligands at pH levels suitable for coupling without appreciable degradation of the ligands.

A second aspect of the invention is to provide a selectively modified sensing surface. This is achieved with a sensing surface as defined in claim 15. One advantage of this surface is that it can be functionalized with pH-sensitive ligands. A further advantage is that it can be functionalized with chemistries requiring mixing of incompatible reagents.

A third aspect of the invention is to provide a flow cell with a plurality of selectively modified sensing surfaces free from cross-talk. This is achieved with a flow cell as defined in claim 17. One advantage is that the different sensing surfaces can be free from the ligands used in the neighbouring surfaces.

A fourth aspect of the invention is to provide a method of detecting a binding event. This is achieved with a method as defined in claim 21.

A fifth aspect of the invention is to provide a method of detecting a plurality of binding events without cross-talk. This is achieved with a method as defined in claim 22.

Further suitable embodiments of the invention are described in the depending claims.

### BRIEF DESCRIPTION OF THE DRAWINGS

FIG. 1 shows a sequence of modifications according to the invention.

FIG. 2 shows a sequence of modifications according to the invention.

FIG. 3 shows a sequence of modifications according to the invention.

FIG. 4 shows removal of an electrostatically bound ligand with sodium hydroxide.

FIG. 5 shows the low degree of coupling with carbodiimide only.

FIG. 6 shows improvements in functionalization using both carbodiimide and an N-hydroxy compound.

### DEFINITIONS

The term “binding agent” as used herein means any agent that is a member of a specific binding pair, including, for instance polypeptides, such as e.g., proteins or fragments thereof, including antibodies; nucleic acids, e.g. oligonucleotides, polynucleotides, and the like; etc. The binding agent is often a ligand.

The term “ligand” as used herein means a molecule that has a known or unknown affinity for a given analyte and can be immobilized on a predestined region of the surface. The ligand may be a naturally occurring molecule or one that has been synthesized. The ligand may be used per se or as aggregates with another species. Optionally, the ligand may also be a cell.

The term “reactive” with respect to a substrate surface herein means that the surface should be amenable to modification with reagents such as activating reagents, deactivating reagents or binding agents. Such modifications may e.g. involve chemical reactions where covalent bonds are formed or broken.

The term “activation” herein means modification of a substrate surface to enable coupling a binding agent thereto, usually modification of a functional group on the substrate surface.

The term “deactivation” herein means modification of a reactive substrate surface, usually of an activated functional group thereon, such that coupling of a binding agent to the surface is substantially prevented. Deactivation may include restoring an original functional group or making a reactive functional group or other reactive moiety inactive in other ways.

The term “coupling” as used herein is to be interpreted in a broad sense and includes covalent binding as well as other types of binding.

### DETAILED DESCRIPTION OF EMBODIMENTS

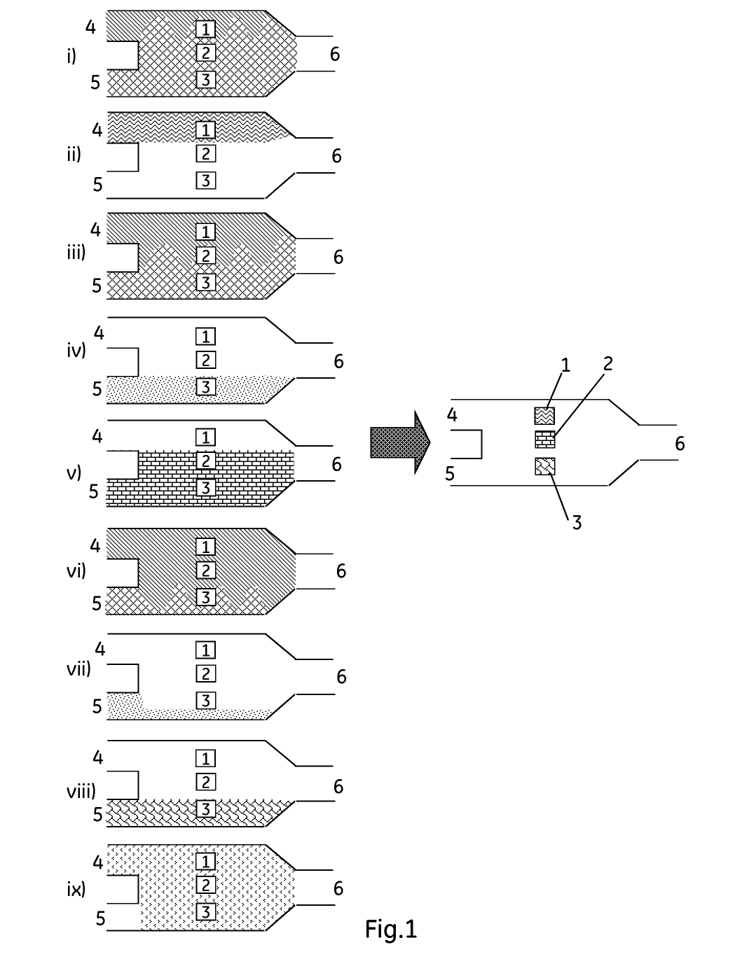

In one aspect illustrated by FIGS. 1-3, the present invention discloses a method of chemically modifying at least one defined area of a substrate surface, comprising the steps of:

a) providing a flow cell having a reactive substrate surface,

b) providing a laminar flow of a first fluid comprising a first reagent and a laminar flow of a second fluid comprising a second reagent, adjacent to the flow of the first fluid, such that the two laminar fluids flow together over the reactive substrate surface with an interface to each other and,

c) dynamically controlling the relative flow rates of the first and second fluids to position the interface such that a predetermined area of the reactive substrate surface is repeatedly exposed to both the first and the second fluid to obtain a chemically modified predetermined area of the substrate surface. In other words, step c) involves dynamical adjustment of the relative flow rates of the two fluids through the flow cell so that a predetermined area of the reactive substrate surface is repeatedly exposed to the interface between the first and the second fluid, resulting in a chemical modification of the predetermined area. The contact line between the fluids will be convoluted and will repeatedly pass over the predetermined area. This increases the diffusive mixing of the two reagents over the interface between the fluids and the repeated exposure of the substrate surface to both fluids also increases the diffusive mixing of the two reagents through diffusion into and out of the surface layer of the reactive substrate surface.

The flow cell can be a Y-type flow cell with two inlets and one outlet as described in e.g. WO 03/102580 and the predetermined area(s) can be arranged along a line perpendicular to the general flow direction of the flow cell. An advantage of repeatedly exposing the surface to both the first and the second fluid is that the two reagents are momentarily mixed and brought into contact with the reactive substrate surface. Hence, any side reactions or degradations occurring between the two reagents will not impair their reactions with the substrate surface.

In some embodiments the predetermined area of the reactive substrate surface is exposed at least five times to the first fluid and at least five times to the second fluid, such as at least twenty times to the first fluid and at least twenty times to the second fluid. This ensures that the diffusion is sufficiently enhanced to provide the desired chemical modification.

In certain embodiments, steps b) and c) are repeated for additional predetermined areas of the reactive substrate surface, wherein in each repetition at least one further reagent is used. The further reagent can be a binding agent and the repetition of steps b) and c) can result in each predetermined area being functionalized with a different binding agent. Steps b) and c) can be repeated at least 1, 2, 3 or 4 times and the substrate surface may comprise at least 2, 3, 4 or 5 predetermined areas functionalized with different binding agents. An advantage of using the repeated exposure technique (step c) when addressing a plurality of predetermined areas with different reagents is that a subsequent reagent does not have to be flowed over an area where a previous reagent has been immobilized. This means that the risk of additional coupling of the subsequent reagent to the area predetermined for the previous reagent is eliminated. Hence, a source of crosstalk between the different areas is avoided. This is particularly important when different binding agents are coupled to more than 3 predetermined areas in a Y-cell.

In some embodiments at least one of said two laminar fluid flows is pulsed, such as with an average pulse frequency from about 0.01 to about 10 Hz or with an average pulse length from about 10 to about 10 000 micrometers or with an average pulse length from about 0.1 to about 100 times the width of the flow cell or of the defined area. A pulsed fluid flow can be obtained by several means known to the skilled person. Pumps can be used to deliver a pulsed flow, e.g.

by using syringe pumps driven by stepper motors that can be programmed to produce pulsing. It is also possible to control pulsations in a flow by valves, e.g. with a valve that intermittently stops, throttles or diverts at least one of the flows. One of the flows can be pulsed while the other is kept constant, but it is also possible to have both flows pulsed. In the latter case, the two flows should be pulsed in counter-phase (with the pulses alternating between the flows).

In certain embodiments, in step a) said reactive substrate surface is an activatable substrate surface, in step b) said first reagent is a first activating reagent and said second reagent is a second activating reagent and in step c) said chemically modified predetermined area is an activated predetermined area. Some activation methods need only one activation reagent, e.g. cyanogen bromide, epichlorohydrin, diepoxides, tresyl chloride, tosyl chloride, divinyl sulfone, dinitrophenol, dipyridyldisulfide etc. Other methods, particularly those starting with carboxyl groups, need both a first and a second activation reagent. Examples of such methods are those giving reactive ester intermediates, such as when using a carbodiimide and an active ester reagent as first and second reagent respectively. The active ester reagent can e.g. be an N-hydroxy compound, pentafluorophenol, 4-sulfo-2,3,5,6-tetrafluorophenol (STP), 2-nitrophenyl-4-sulfonic acid sodium salt (NFSSNA) or 4-nitrophenol.

In some embodiments, the modification method further comprises a step d) of providing a flow of a third fluid comprising a binding agent over said activated predetermined area of the substrate surface to couple said binding agent to said activated predetermined area. In certain embodiments, steps b), c) and d) are repeated for additional predetermined areas of the reactive substrate surface, wherein in each repetition at least one further binding agent is used in step d). If different binding agents are used in each repetition, the result will be a substrate surface with predetermined areas functionalized with different binding agents. Steps b), c) and d) can be repeated at least 1, 2, 3 or 4 times and the substrate surface may comprise at least 2, 3, 4 or 5 predetermined areas functionalized with different binding agents. One of the predetermined areas can in step d) be reacted with a deactivating agent instead of a binding agent in order to create a non-binding reference predetermined area.

In certain embodiments said activatable substrate surface comprises carboxyl groups, said first activating reagent comprises a carbodiimide and/or said second activating reagent comprises an N-hydroxy compound. The activatable substrate surface can in some embodiments comprise a swellable layer, such as a carboxymethyl dextran layer, e.g. coupled to a self-assembled layer on a gold surface as described in U.S. Pat. No. 5,242,828. An advantage of having a swellable layer is that diffusion into and out of the layer will enhance mixing of reagents when the two fluids are repeatedly brought into contact with the layer. As the O-acylisourea esters formed by reaction between the carboxylic acid and the carbodiimide are highly unstable, it is advantageous if they can directly after formation be exposed to the N-hydroxy compound, which creates a more stable activated ester.

In some embodiments the carbodiimide is selected from the group consisting of ethyldimethylaminopropylcarbodiimide (EDC), diisopropylcarbodiimide (DIPC), dicyclohexylcarbodiimide (DCC), cyclohexylmorpholinoethylcarbodiimide (CMC), N-tert-butyl-N′-methylcarbodiimide (TBMC), and N-tert-butyl-N′-ethylcarbodiimide (TBEC). If high water solubility of the reagents is desired, the carbodiimide can be selected from the group consisting of EDC and CMC.

In certain embodiments the N-hydroxy compound is selected from the group consisting of N-hydroxysuccinimide (NHS), N-hydroxysulfosuccinimide, and N-hydroxybenzotriazolohydrate.

The choice of deactivating agent depends on the active group(s) to be deactivated. For example, N-hydroxysuccinimide esters prepared by activation with a carbodiimide plus NHS may be deactivated with ethanolamine or sodium hydroxide, deactivation with sodium hydroxide being reversible, i.e. the deactivated surface may be reactivated by activation with an activating agent.

In certain embodiments, in step a) said reactive substrate surface is an activated substrate surface, in step b) said first reagent is a binding agent and said second reagent is a pH-regulating compound and in step c) said chemically modified predetermined area is a predetermined area with immobilized binding agent. The activated substrate surface can be activated using any activation method suitable for the functional groups on the surface, using either a method with just one activation reagent or a method with a first and a second activation reagent. Many binding agents, such as e.g. proteinaceous ligands are sensitive to pH and may be inactivated by long-time exposure to the conditions needed for efficient coupling to the activated surface. Some coupling methods require acidic pH levels (e.g. coupling on N-hydroxysuccinimide esters), while other methods require basic pH levels, e.g. coupling of aminofunctional ligands on epoxide groups. In both cases, sensitive proteins, e.g. antibodies, or other binding agents can suffer damage. With the method of the invention it is possible to apply the sensitive binding agent at a pH where it is stable and in a separate flow supply a pH-regulating compound to produce a suitable coupling pH directly at the activated substrate surface and thus reduce the exposure time for the sensitive binding agent to this pH to an absolute minimum. The pH-regulating compound can comprise a buffering salt, a base and/or an acid.

In certain embodiments said binding agent is unstable at pH 5 or lower and said second fluid has a pH less than 5. One example of coupling methods requiring a pH below 5 is the coupling of proteins on N-hydroxysuccinimide esters and examples of typical proteins sensitive to these conditions are anti-GST antibodies, PARP (poly ADP ribose polymerase), TNKS (TRF1-interacting ankyrin-related ADP-ribose polymerase), carbanhydrase and p38 (a mitogen-activated protein kinase). In certain embodiments the substrate surface comprises a swellable layer, such as a carboxymethyl dextran layer, with N-hydroxysuccinimide esters of carboxyl groups.

In some embodiments, steps b) and c) are repeated for additional predetermined areas of the reactive substrate surface, wherein in each repetition at least one further binding agent is used in step b). If different binding agents are used in each repetition, the result will be a substrate surface with predetermined areas functionalized with different binding agents. Steps b) and c) can be repeated at least 1, 2, 3 or 4 times and the substrate surface may comprise at least 2, 3, 4 or 5 predetermined areas functionalized with different binding agents. One of the predetermined areas can in step b) be reacted with a deactivating agent instead of a binding agent in order to create a non-binding reference predetermined area.

In a second aspect, the present invention discloses a sensing surface, which has been prepared according to any of the methods disclosed above. The term sensing surface in the present context is to be construed broadly. The sensing surface may, for example, be a surface or surface layer that can interact specifically with a species present in a fluid, a surface or surface layer that can be chemically or physically sensitised to permit such interaction, or a surface or surface layer that can be chemically or physically activated to permit sensitization thereof. When a sensing surface is functionalized with a binding agent, it is possible to detect a binding event wherein a species in a sample binds to the binding agent. The detection of a binding event can be used for determining the concentration of the species in the sample or to analyze the binding strength and/or interaction kinetics between the species and the binding agent. Binding events at the sensing surface may be detected by numerous techniques.

In many cases it is favourable to use so-called non-label methods. Representative such detection methods include, but are not limited to, mass detection methods, such as piezoelectric, optical, thermo-optical and surface acoustic wave (SAW) device methods, and electrochemical methods, such as potentiometric, conductometric, amperometric and capacitance/impedance methods. With regard to optical detection methods, representative methods include those that detect mass surface concentration, such as reflection-optical methods, including both external and internal reflection methods, angle, wavelength, polarization, or phase resolved, for example evanescent wave ellipsometry and evanescent wave spectroscopy (EWS, or internal reflection spectroscopy), both may include evanescent field enhancement via surface plasmon resonance (SPR), Brewster angle refractometry, critical angle refractometry, frustrated total reflection (FTR), scattered total internal reflection (STIR), which may include scatter enhancing labels, optical wave guide sensors; external reflection imaging, evanescent wave-based imaging such as critical angle resolved imaging, Brewster angle resolved imaging, SPR-angle resolved imaging, and the like. Further, photometric and imaging/microscopy methods, “per se” or combined with reflection methods, based on for example surface enhanced Raman spectroscopy (SERS), surface enhanced resonance Raman spectroscopy (SEARS), evanescent wave fluorescence (TIRE) and phosphorescence may be mentioned, as well as waveguide interferometers, waveguide leaking mode spectroscopy, reflective interference spectroscopy (RIfS), transmission interferometry, holographic spectroscopy, and atomic force microscopy (AFR).

SPR spectroscopy may be mentioned as an exemplary commercially available analytical system to which the present invention may be applied. One type of SPR based biosensors is sold by GE Healthcare Bio-Sciences AB (Uppsala, Sweden) under the trade name BIACORE. These biosensors utilize a SPR based mass-sensing technique to provide a “real-time” binding interaction analysis between a surface bound ligand and an analyte of interest.

In some embodiments, the sensing surface has at least one lateral dimension within the interval 1 micrometer-500 micrometers, such as 10-200, 10-150 or 50-100 micrometers.

In certain embodiments, the sensing surface is functionalized with pH-sensitive ligands. Such ligands can e.g. be proteins that denature to an extent of at least 10% in buffers of pH 5.0 or lower for 30 minutes at 25 degrees C. Examples of pH-sensitive ligands are anti-GST antibodies, PARP (poly ADP ribose polymerase), TNKS (TRF1-interacting ankyrin-related ADP-ribose polymerase), carbanhydrase and p38 (a mitogen-activated protein kinase).

In a third aspect, the present invention discloses a flow cell comprising a plurality of sensing surfaces that have been functionalized with different binding agents according to the methods described above. A flow cell may comprise one or more sensing surfaces, such as at least 2, 3, 4 or 5 sensing surfaces functionalized with different binding agents. If a flow cell comprises several sensing surfaces functionalized with different binding agents it is possible to detect several binding events simultaneously.

In some embodiments, each sensing surface has at least one lateral dimension within the interval 1 micrometer-500 micrometers, such as 10-200, 10-150 or 50-100 micrometers.

In certain embodiments, the flow cell comprises at least 4 separate sensing surfaces, such as at least 5, 4-10 or 5-10 separate sensing surfaces.

In some embodiments, the smallest lateral distance between any neighbouring sensing surfaces is within the interval 1 micrometer-500 micrometers, such as 10-200, 10-150 or 50-100 micrometers.

In a fourth aspect, the present invention discloses a method of detecting a binding event on a sensing surface that has been chemically modified according to the methods described above.

In a fifth aspect, the present invention discloses a method of detecting a plurality of binding events in a flow cell as described above.

### EXAMPLES

### Example 1

### Two Activation Reagents in Pulsed Flow

This example, illustrated by FIG. 1, uses a Y-cell with two inlets 4,5, one outlet 6 and three discrete SPR sensing areas 1,2,3, each with a carboxymethyl dextran layer on top of a self-assembled thiol layer anchored on a gold film as described in U.S. Pat. No. 5,242,828. The flow-path in the Y-cell has a width of 900 micrometers and a height of 45 micrometers. The Y-cell is mounted in a BIACORE 4000 or BIACORE A100 instrument (GE Healthcare Bio-Sciences AB, Sweden), equipped with two stepper motor driven syringe pumps capable of delivering flow rates of 2.5-50 microliters/min to each inlet with either constant or pulsed flow rates.

i) An aqueous solution of 0.2 M NHS is pumped via inlet 4 at a pulsed flow of 0.1 Hz pulse frequency. An aqueous solution of 0.4 M EDC is pumped via inlet 5 at a pulsed flow of 0.1 Hz pulse frequency with the pulses in counter-phase to the NHS pulses and the relative flow rates and pulse amplitudes matched so that sensing area 1 is repeatedly exposed to both NHS and EDC, while sensing areas 2 and 3 are only exposed to EDC. This step continues for 10 mM and activates area 1 only.

ii) A solution of 50 micrograms/ml protein ligand 1 is pumped via inlet 4 at a constant flow rate. An HBS-N running buffer (10 mM HEPES pH 7.4 and 150 mM NaCl) is pumped via inlet 5 at a constant flow rate, with the relative flow rates matched so that sensing area 1 is exposed to protein ligand 1, while sensing areas 2 and 3 are exposed to the running buffer. This step continues for 7 mM and couples protein ligand 1 to area 1 only.

iii) An aqueous solution of 0.2 M NHS is pumped via inlet 4 at a pulsed flow of 0.1 Hz pulse frequency. An aqueous solution of 0.4 M EDC is pumped via inlet 5 at a pulsed flow of 0.1 Hz pulse frequency with the pulses in counter-phase to the NHS pulses and the relative flow rates and pulse amplitudes matched so that sensing area 1 is only exposed to NHS, sensing area 2 is repeatedly exposed to both NHS and EDC, while sensing area 3 is only exposed to EDC. This step continues for 10 mM and activates area 2, with some degree of activation in area 3 also.

iv) A solution of 50 mM NaOH and 0.1 M NaCl is pumped via inlet 5 at a constant flow rate. An HBS-N running buffer is pumped via inlet 4 at a constant flow rate, with the relative flow rates matched so that sensing areas 1 and 2 are exposed to running buffer, while sensing area 3 is exposed to the NaOH solution. This step continues for 2 min and deactivates area 3 only.

v) A solution of 50 micrograms/ml protein ligand 2 is pumped via inlet 5 at a constant flow rate. An HBS-N running buffer is pumped via inlet 4 at a constant flow rate, with the relative flow rates matched so that sensing area 1 is exposed to running buffer, while sensing areas 2 and 3 are exposed to protein ligand 2. This step continues for 7 mM and couples protein ligand 2 to area 2 only.

vi) An aqueous solution of 0.2 M NHS is pumped via inlet 4 at a pulsed flow of 0.1 Hz pulse frequency. An aqueous solution of 0.4 M EDC is pumped via inlet 5 at a pulsed flow of 0.1 Hz pulse frequency with the pulses in counter-phase to the NHS pulses and the relative flow rates and pulse amplitudes matched so that sensing areas 1 and 2 are only exposed to NHS, and sensing area 3 is repeatedly exposed to both NHS and EDC. This step continues for 10 mM and activates area 3 only.

vii) An HBS-N running buffer is pumped via inlet 4 at a constant flow rate. A solution of 50 mM NaOH and 0.1 M NaCl is pumped via inlet 5 at a constant flow rate, with the relative flow rates matched so that sensing areas 1, 2 and 3 are exposed to running buffer, while the area beyond sensing area 3 is exposed to the NaOH solution. This step (optional) continues for 2 mM and deactivates the area outside sensing area 3.

viii) An HBS-N running buffer is pumped via inlet 4 at a constant flow rate. A solution of 50 micrograms/ml protein ligand 3 is pumped via inlet 5 at a constant flow rate, with the relative flow rates matched so that sensing areas 1 and 2 are exposed to running buffer, while sensing area 3 is exposed to protein ligand 3. This step continues for 7 mM and couples protein ligand 3 to area 3 only.

ix) A solution of 0.1 M ethanolamine is pumped via inlet 4 at a constant flow rate. This step continues for 2 mM and endcaps any remaining active groups.

### Example 2

### Binding Agent and Acidic Buffer in Pulsed Flow

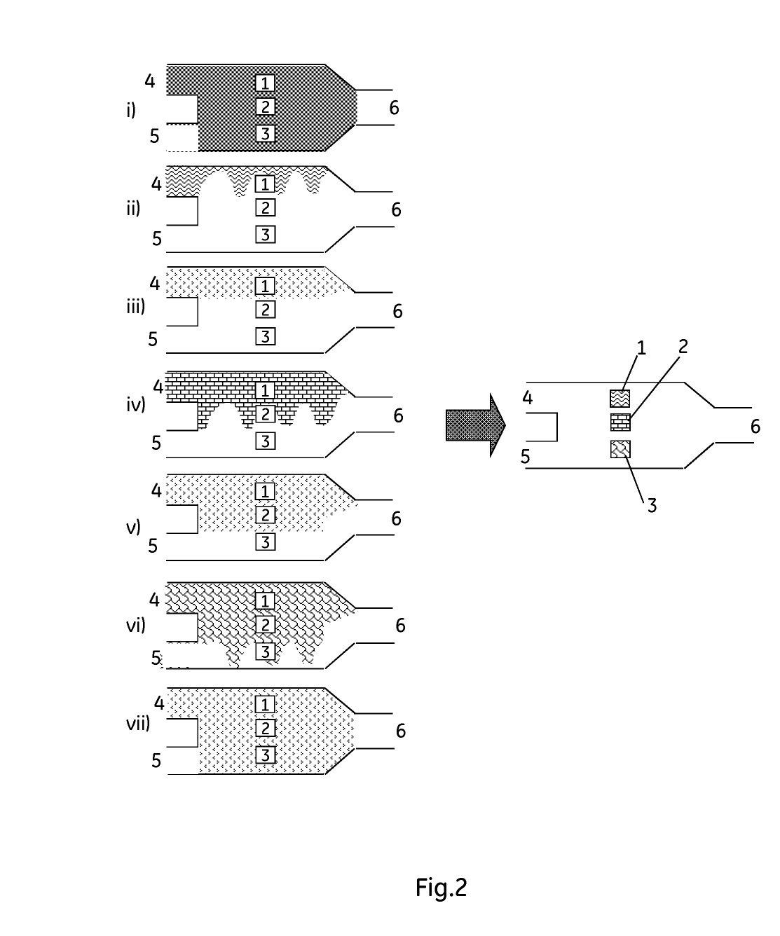

This example, illustrated by FIG. 2, uses the same type of Y-cell and setup as in Example 1.

i) An aqueous solution of 0.2 M NHS and 0.4 M EDC is pumped via inlet 4 at a constant flow rate. This step continues for 10 mM and activates all three sensing areas.

ii) A solution of 50 micrograms/ml acid-sensitive protein ligand 1a at pH 7 is pumped via inlet 4 at a pulsed flow flow of 0.1 Hz pulse frequency. A buffer of pH 4.5 is pumped via inlet 5 at a pulsed flow of 0.1 Hz pulse frequency with the pulses in counter-phase to the protein ligand pulses and the relative flow rates and pulse amplitudes matched so that sensing area 1 is repeatedly exposed to both protein ligand 1a and pH 4.5 buffer, while sensing areas 2 and 3 are only exposed to pH 4.5 buffer. This step continues for 7 min and couples acid-sensitive protein ligand1 1a to sensing area 1 only.

iii) A solution of 0.1 M ethanolamine is pumped via inlet 4 at a constant flow rate. A buffer of pH 4.5 is pumped via inlet 5 at a constant flow rate, with the relative flow rates matched so that sensing area 1 is exposed to ethanolamine, while sensing areas 2 and 3 are exposed to the pH 4.5 buffer. This step continues for 2 min and endcaps any remaining active groups in sensing area 1.

iv) A solution of 50 micrograms/ml acid-sensitive protein ligand 2a at pH 7 is pumped via inlet 4 at a pulsed flow flow of 0.1 Hz pulse frequency. A buffer of pH 4.5 is pumped via inlet 5 at a pulsed flow of 0.1 Hz pulse frequency with the pulses in counter-phase to the protein ligand pulses and the relative flow rates and pulse amplitudes matched so that sensing area 1 is only exposed to protein ligand 2a, sensing area 2 is repeatedly exposed to both protein ligand 2a and pH 4.5 buffer, while sensing area 3 is only exposed to pH 4.5 buffer. This step continues for 7 min and couples acid-sensitive protein ligand 2a to sensing area 2 only.

v) A solution of 0.1 M ethanolamine is pumped via inlet 4 at a constant flow rate. A buffer of pH 4.5 is pumped via inlet 5 at a constant flow rate, with the relative flow rates matched so that sensing areas 1 and 2 are exposed to ethanolamine, while sensing area 3 is exposed to the pH 4.5 buffer. This step continues for 2 min and endcaps any remaining active groups in sensing area 2.

vi) A solution of 50 micrograms/ml acid-sensitive protein ligand 3a at pH 7 is pumped via inlet 4 at a pulsed flow flow of 0.1 Hz pulse frequency. A buffer of pH 4.5 is pumped via inlet 5 at a pulsed flow of 0.1 Hz pulse frequency with the pulses in counter-phase to the protein ligand pulses and the relative flow rates and pulse amplitudes matched so that sensing areas 1 and 2 are only exposed to protein ligand 3a, while sensing area 3 is repeatedly exposed to both protein ligand 3a and pH 4.5 buffer. This step continues for 7 min and couples acid-sensitive protein ligand 3a to sensing area 3 only.

vii) A solution of 0.1 M ethanolamine is pumped via inlet 4 at a constant flow rate. This step continues for 2 min and endcaps any remaining active groups in sensing area 3.

### Example 3

### Two Activation Reagents in Pulsed Flow Plus Binding Agent and Acidic Buffer in Pulsed Flow

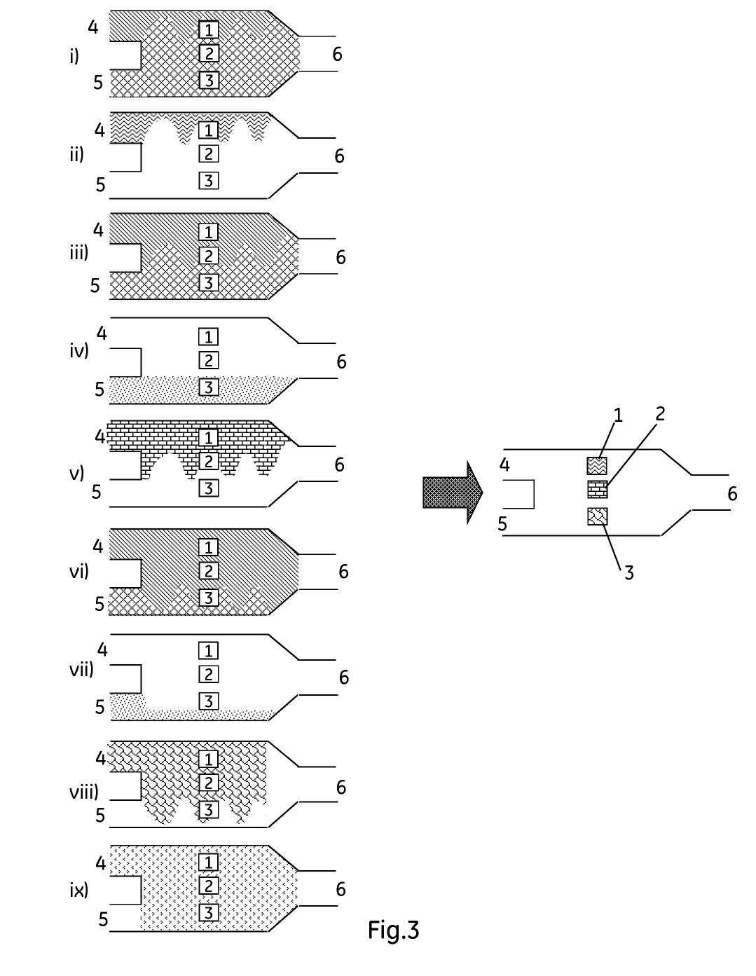

This example, illustrated by FIG. 3, uses the same type of Y-cell and setup as in Examples 1 and 2.

i) An aqueous solution of 0.2 M NHS is pumped via inlet 4 at a pulsed flow of 0.1 Hz pulse frequency. An aqueous solution of 0.4 M EDC is pumped via inlet 5 at a pulsed flow of 0.1 Hz pulse frequency with the pulses in counter-phase to the NHS pulses and the relative flow rates and pulse amplitudes matched so that sensing area 1 is repeatedly exposed to both NHS and EDC, while sensing areas 2 and 3 are only exposed to EDC. This step continues for 10 mM and activates area 1 only.

ii) A solution of 50 micrograms/ml acid-sensitive protein ligand 1a at pH 7 is pumped via inlet 4 at a pulsed flow flow of 0.1 Hz pulse frequency. A buffer of pH 4.5 is pumped via inlet 5 at a pulsed flow of 0.1 Hz pulse frequency with the pulses in counter-phase to the protein ligand pulses and the relative flow rates and pulse amplitudes matched so that sensing area 1 is repeatedly exposed to both protein ligand 1a and pH 4.5 buffer, while sensing areas 2 and 3 are only exposed to pH 4.5 buffer. This step continues for 7 mM and couples acid-sensitive protein ligand 1a to sensing area 1 only.

iii) An aqueous solution of 0.2 M NHS is pumped via inlet 4 at a pulsed flow of 0.1 Hz pulse frequency. An aqueous solution of 0.4 M EDC is pumped via inlet 5 at a pulsed flow of 0.1 Hz pulse frequency with the pulses in counter-phase to the NHS pulses and the relative flow rates and pulse amplitudes matched so that sensing area 1 is only exposed to NHS, sensing area 2 is repeatedly exposed to both NHS and EDC, while sensing area 3 is only exposed to EDC. This step continues for 10 mM and activates area 2, with some degree of activation in area 3 also.

iv) A solution of 50 mM NaOH and 0.1 M NaCl is pumped via inlet 5 at a constant flow rate. An HBS-N running buffer is pumped via inlet 4 at a constant flow rate, with the relative flow rates matched so that sensing areas 1 and 2 are exposed to running buffer, while sensing area 3 is exposed to the NaOH solution. This step continues for 2 mM and deactivates area 3 only.

v) A solution of 50 micrograms/ml acid-sensitive protein ligand 2a at pH 7 is pumped via inlet 4 at a pulsed flow flow of 0.1 Hz pulse frequency. A buffer of pH 4.5 is pumped via inlet 5 at a pulsed flow of 0.1 Hz pulse frequency with the pulses in counter-phase to the protein ligand pulses and the relative flow rates and pulse amplitudes matched so that sensing area 1 is only exposed to protein ligand 2a, sensing area 2 is repeatedly exposed to both protein ligand 2a and pH 4.5 buffer, while sensing area 3 is only exposed to pH 4.5 buffer. This step continues for 7 mM and couples acid-sensitive protein ligand 2a to sensing area 2 only.

vi) An aqueous solution of 0.2 M NHS is pumped via inlet 4 at a pulsed flow of 0.1 Hz pulse frequency. An aqueous solution of 0.4 M EDC is pumped via inlet 5 at a pulsed flow of 0.1 Hz pulse frequency with the pulses in counter-phase to the NHS pulses and the relative flow rates and pulse amplitudes matched so that sensing areas 1 and 2 are only exposed to NHS, and sensing area 3 is repeatedly exposed to both NHS and EDC. This step continues for 10 mM and activates area 3 only.

vii) An HBS-N running buffer is pumped via inlet 4 at a constant flow rate. A solution of 50 mM NaOH and 0.1 M NaCl is pumped via inlet 5 at a constant flow rate, with the relative flow rates matched so that sensing areas 1, 2 and 3 are exposed to running buffer, while the area beyond sensing area 3 is exposed to the NaOH solution. This step (optional) continues for 2 mM and deactivates the area outside sensing area 3.

viii) A solution of 50 micrograms/ml acid-sensitive protein ligand 3a at pH 7 is pumped via inlet 4 at a pulsed flow flow of 0.1 Hz pulse frequency. A buffer of pH 4.5 is pumped via inlet 5 at a pulsed flow of 0.1 Hz pulse frequency with the pulses in counter-phase to the protein ligand pulses and the relative flow rates and pulse amplitudes matched so that sensing areas 1 and 2 are only exposed to protein ligand 3a, while sensing area 3 is repeatedly exposed to both protein ligand 3a and pH 4.5 buffer. This step continues for 7 min and couples acid-sensitive protein ligand 3a to sensing area 3 only.

ix) A solution of 0.1 M ethanolamine is pumped via inlet 4 at a constant flow rate. This step continues for 2 mM and endcaps any remaining active groups in sensing area 3.

### Example 4

### Non-Specific Binding to Unactivated Carboxyl Surface

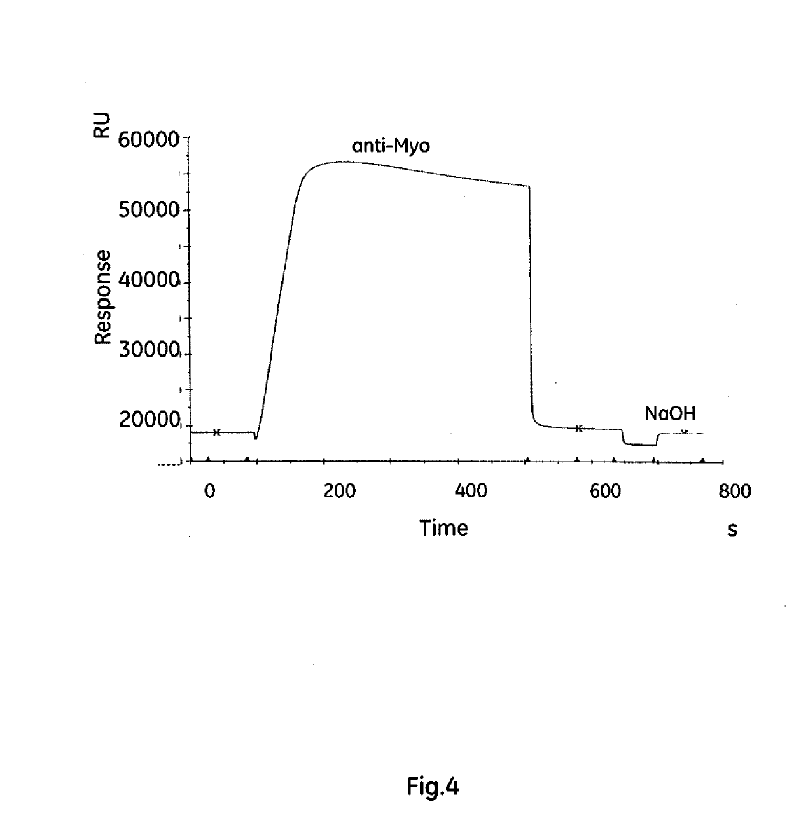

A BIACORE Sensor Chip CM5 (GE Healthcare Bio-Sciences, Sweden) with a carboxymethyl dextran surface anchored on a gold layer was mounted in a flow cell with one inlet and one outlet (length 2.4 mm, width 0.5 mm, height 0.002 mm) and fitted in a BIACORE 3000 SPR instrument. A solution of 50 micrograms/ml anti-myglobulin antibodies in a pH 4.5 buffer was injected at a flow rate of 10 microliters/min and the SPR response (level of binding) was recorded. A solution of 50 mM NaOH was then injected while recording the SPR response. As shown in FIG. 4, a significant amount of antibody was bound to the carboxyl surface, but was completely removed by the NaOH wash, which shows that it was not covalently coupled.

### Example 5

### Low Degree of Coupling with EDC-Only Activation

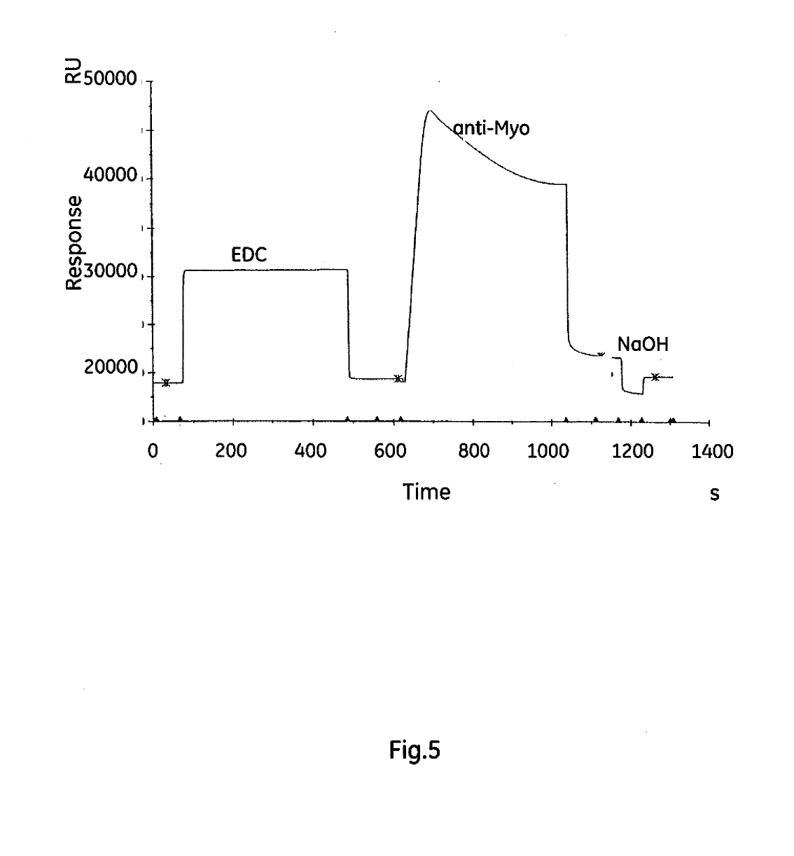

A BIACORE Sensor Chip CM5 (GE Healthcare Bio-Sciences, Sweden) with a carboxymethyl dextran surface anchored on a gold layer was mounted in a flow cell with one inlet and one outlet (length 2.4 mm, width 0.5 mm, height 0.002 mm) and fitted in a BIACORE 3000 SPR instrument. An aqueous solution of 0.4 M EDC was injected at a flow rate of 10 microliters/min and the SPR response (level of binding) was recorded. After a wash, a solution of 50 micrograms/ml anti-myglobulin antibodies in a pH 4.5 buffer was injected while recording the SPR response. Finally, a solution of 50 mM NaOH was injected. As shown in FIG. 5, most of the bound antibody was removed by the NaOH wash, which shows that only a very low degree of covalent coupling occurred when EDC was used as the single activation reagent.

### Example 6

### Coupling with both EDC and NHS

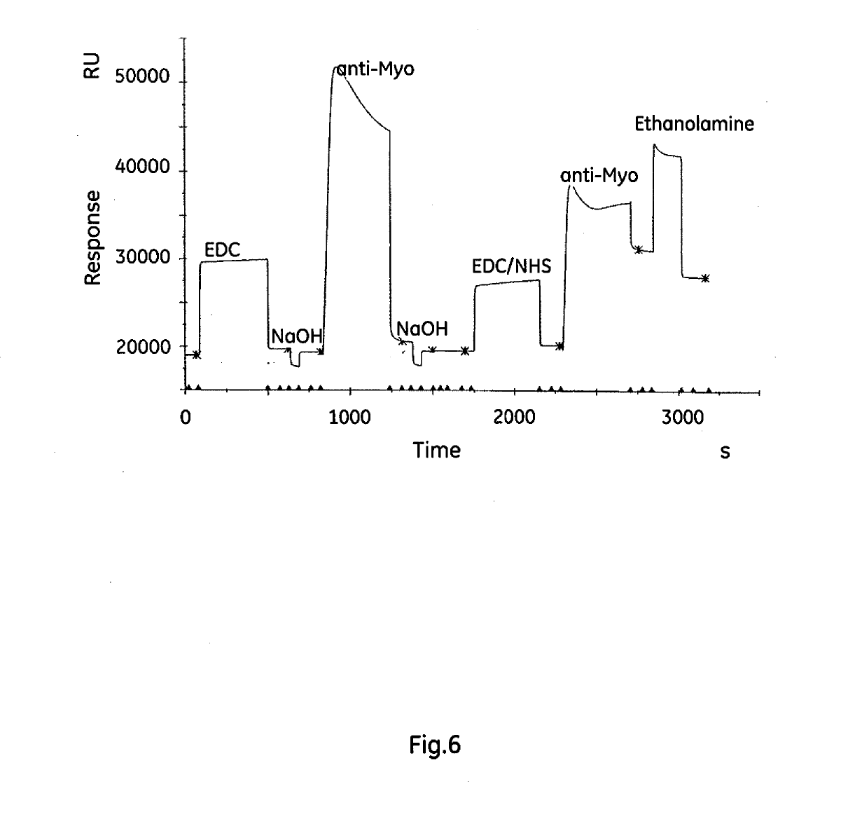

A BIACORE Sensor Chip CM5 (GE Healthcare Bio-Sciences, Sweden) with a carboxymethyl dextran surface anchored on a gold layer was mounted in a flow cell with one inlet and one outlet (length 2.4 mm, width 0.5 mm, height 0.002 mm) and fitted in a BIACORE 3000 SPR instrument. An aqueous solution of 0.4 M EDC was injected at a flow rate of 10 microliters/min and the SPR response (level of binding) was recorded. After a wash with 50 mM NaOH, a solution of 50 micrograms/ml anti-myglobulin antibodies in a pH 4.5 buffer was injected while recording the SPR response. After another 50 mM NaOH wash, a solution of 0.4 M EDC+0.2 M NHS was injected, followed by a solution of 50 micrograms/ml anti-myglobulin antibodies in a pH 4.5 buffer and a solution of 0.1 M ethanolamine. As shown in FIG. 6, the presence of both EDC and NHS gave a much higher degree of coupling to the surface.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. It is pointed out that any feature described in relation to one embodiment may be used also in combination with one or more features of any other of the aspects and embodiments described.

1. A method of chemically modifying at least one defined area of a substrate surface, comprising the steps of:
a) providing a flow cell having a reactive substrate surface;
b) providing a laminar flow of a first fluid comprising a first reagent and a laminar flow of a second fluid comprising a second reagent, adjacent to the flow of the first fluid, such that the two laminar fluids flow together over the reactive substrate surface with an interface to each other; and
c) dynamically controlling the relative flow rates of the first and second fluids to position the interface so that a predetermined area of the reactive substrate surface is repeatedly exposed to both the first and the second fluid to obtain a chemically modified predetermined area of the substrate surface.

2. The method of claim 1, wherein the predetermined area of the reactive substrate surface is exposed at least five times to the first fluid and at least five times to the second fluid, such as at least twenty times to the first fluid and at least twenty times to the second fluid.

3. The method of claim 1, wherein steps b) and c) are repeated for additional predetermined areas of the reactive substrate surface and wherein in each repetition at least one further reagent is used.

4. The method of claim 1, wherein at least one of said two laminar fluid flows is pulsed.

5. The method of claim 4, wherein the average pulse frequency is from about 0.01 to about 10 Hz, the average pulse length is from about 10 to about 10 000 micrometers or the average pulse length is from about 0.1 to about 100 times the width of the predetermined area.

6. The method of claim 1, wherein in step a) said reactive substrate surface is an activatable substrate surface, in step b) said first reagent is a first activating reagent and said second reagent is a second activating reagent and in step c) said chemically modified predetermined area is an activated predetermined area.

7. The method of claim 6, further comprising step d) providing a flow of a third fluid comprising a binding agent over said activated predetermined area of the substrate surface to couple said binding agent to said activated predetermined area.

8. The method of claim 6, wherein steps b), c) and d) are repeated for additional predetermined areas of the reactive substrate surface, wherein in each repetition at least one further binding agent is used in step d).

9. The method of claim 7, wherein said activatable substrate surface comprises carboxyl groups, said first activating reagent comprises a carbodiimide and/or said second activating reagent comprises an N-hydroxy compound.

10. The method of claim 9, wherein said carbodiimide is selected from the group consisting of ethyldimethylaminopropylcarbodiimide (EDC), diisopropylcarbodiimide (DIPC), dicyclohexylcarbodiimide (DCC), cyclohexylmorpholinoethylcarbodiimide (CMC), N-tert-butyl-N′-methylcarbodiimide (TBMC), and N-tert-butyl-N′-ethylcarbodiimide (TBEC).

11. The method of claim 10, wherein said N-hydroxy compound is selected from the group consisting of N-hydroxysuccinimide (NHS), hydroxysulfosuccinimide, and hydroxybenzotriazolohydrate.

12. The method of claim 1, wherein in step a) said reactive substrate surface is an activated substrate surface, in step b) said first reagent is a binding agent and said second reagent is a pH-regulating compound and in step c) said chemically modified predetermined area is a predetermined area with immobilized binding agent.

13. The method of claim 12, wherein said binding agent is unstable at pH 5 or lower and said second fluid has a pH less than 5.

14. The method of claim 12, wherein steps b) and c) are repeated for additional predetermined areas of the reactive substrate surface, wherein in each repetition at least one further binding agent is used in step b)

15. A sensing surface prepared according to claim 1.

16. The sensing surface of claim 15, having at least one lateral dimension within the interval 1 micrometer-500 micrometers, such as 10-200 or 50-100 micrometers.

17. A flow cell comprising a plurality of sensing surfaces that have been functionalized with different binding agents of claim 3.

18. The flow cell of claim 17, wherein each sensing surface has at least one lateral dimension within the interval within the interval 1 micrometer-500 micrometers, such as 10-200 or 50-100 micrometers.

19. The flow cell of claim 17, comprising at least 4 separate sensing surfaces, such as at least 5, 4-10 or 5-10 separate sensing surfaces.

20. The flow cell of claim 17, wherein the smallest lateral distance between any neighbouring sensing surfaces is within the interval 1 micrometer-500 micrometers, such as 10-200, 10-150 or 50-100 micrometers.

21. A method of detecting a binding event on a sensing surface that has been chemically modified according to claim 1.

22. A method of detecting a plurality of binding events in a flow cell according to claim 18.
